(19) United States
(12) Patent Application Publication
(10) Pub. No.: US 2022/0184216 A1
(43) Pub. Date: Jun. 16, 2022

(54) INTRACELLULAR TARGETING OF MOLECULES

(71) Applicant: Hoffmann-La Roche Inc., Little Falls, NJ (US)

(72) Inventors: Ashwath JAYAGOPAL, Mountain View, CA (US); Kameron V. KILCHRIST, San Diego, CA (US); Martin G. NUSSBAUMER, Bern (CH)

(73) Assignee: Hoffmann-La Roche Inc., Little Falls, NJ (US)

(21) Appl. No.: 17/436,451

(22) PCT Filed: Mar. 3, 2020
(86) PCT No.: PCT/EP2020/055480

§ 371 Date: Sep. 3, 2021

Related U.S. Application Data
(60) Provisional application No. 62/813,833, filed on Mar. 5, 2019.

(51) Int. Cl. A61K 47/54; C12N 15/113

(52) U.S. Cl. CPC A61K 47/545; C12N 15/113; C12N 2310/351; C12N 2310/11; C12N 2310/341; C12N 2310/315; C12N 2310/3231

(57) ABSTRACT

The present invention relates to conjugates comprising a small molecule targeting ligand and a cargo molecule.

The present invention relates to conjugates comprising a small molecule ligand and a cargo molecule to be delivered intracellularly.

Biomacromolecules are naturally occurring or synthetic heteropolymers of nucleic acids, amino acids, synthetic derivatives of nucleic acids, or synthetic derivatives of amino acids. Examples of biopolymers include proteins, antibodies, genomic DNA (gDNA), messenger RNA (mRNA), small interfering RNA (siRNA), antisense oligonucleotides (ASOs), oligodeoxynucleotides (ODNs), and locked nucleic acids (LNAs).

LNA antisense oligonucleotides are synthetic oligonucleotides consisting of a mixture of DNA and an RNA modified with, e.g., a 2′-0,4′-C-methylene bridge. LNAs can be used to modulate mRNA splicing, to effect exon skipping, to effect RNAse-H mediated mRNA degradation, and to reduce mRNA translation to protein by complementary base pairing.

Biomacromolecules are attractive as medicines due to their bioactivity or use as a template or substrate for enzymatic activity, but the development of this class of therapy is limited by poor pharmacokinetics due to limited access to the intracellular space.

A number of approaches have been used to improve bioactivity of biomacromolecules as intracellular therapies, such as viruses, polymers, lipids, viruses, and small molecule targeting ligands.

Small molecule targeting ligands represent a particularly attractive approach due to their facile chemical synthesis and modular architecture. Some targeting ligands which have been explored include folate, N-acetylgalactosamine, and others, which cause the biomacromolecule-ligand complex to bind to a cell receptor, in some cases triggering internalization. This approach can be of limited utility due to low affinity between the small molecule ligand and the receptor. Improvements in ligand receptor affinity can come from alterations to the small molecule ligand or the engineering of a multivalent ligands to improve affinity by exploiting avidity effects.

Therefore, there is a need for a delivery system enabling the transport of therapeutically active molecules into cells.

### BRIEF SUMMARY OF THE INVENTION

In a first aspect, the present invention relates to a conjugate comprising a small molecule targeting ligand and a cargo molecule having formula I:

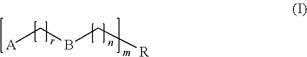

wherein A is selected from the group consisting of:

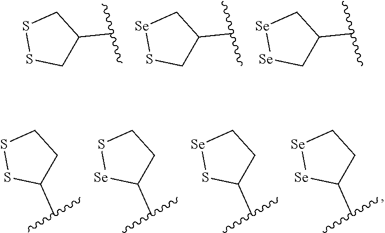

B is —C(O)—O— or —C(O)—N—,

n is selected from 0 or 1-6,

r is selected from 0 or 1-6

m is selected from 1-6, and

R is a cargo molecule.

In a particular embodiment of the invention the cargo molecule is selected from the group of peptides, polypeptides, oligonucleotides.

In a particular embodiment of the invention the cargo molecule is an antibody or an oligonucleotide.

In a particular embodiment of the invention the cargo molecule is a LNA oligonucleotide.

In a particular embodiment of the invention, the small molecule targeting ligand is linked to the oligonucleotide at its 3′ end or 5′ end, preferably at its 5′ end.

In a particular embodiment of the invention, B is —C(O)—N—.

In a particular embodiment of the invention, r=0 and n=6.

In a particular embodiment of the invention, m is selected from 1-4.

In a particular embodiment of the invention, A is

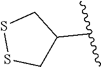 [SMILES: *C1CSSC1]

In a particular embodiment of the invention A is selected from the group consisting of:

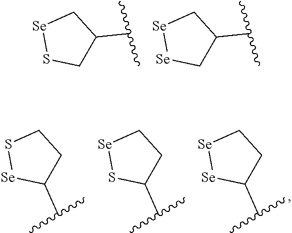

In a particular embodiment of the invention A is selected from the group consisting of:

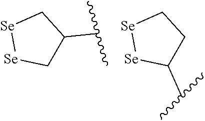

In a particular embodiment of the invention A is selected from the group consisting of:

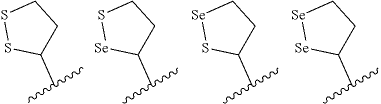

In a particular embodiment of the invention A is

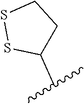 [SMILES: *C1CCSS1]

In a particular embodiment of the invention, the conjugate has the structure given in formula II:

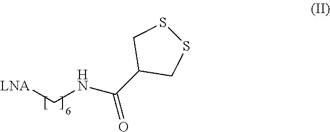

In a second aspect, the present invention relates to a pharmaceutical composition comprising a conjugate of the present invention and a pharmaceutically acceptable carrier.

In a particular embodiment of the invention, the pharmaceutical formulation is a topical composition for eye conditions.

In a third aspect the present invention relates to a method of treating an individual having an eye condition comprising administering to the eye of the individual an effective amount of the conjugate of the present invention or the pharmaceutical composition of the present invention.

### BRIEF DESCRIPTION OF THE DRAWINGS

FIG. 1: LNA modification with 1,2-dithiolane-4-carboxylic acid enhances cellular uptake, quantified by fluorescence microscopy. LNAs labeled with fluorescein isothiocyanate (FITC) were either used unmodified or conjugated to AspA. Cells were treated at indicated doses and washed per “Cellular Uptake Methods”. FIG. 1A) Fluorescence of images was quantified and plotted; the x-axis indicates dose of LNA, in nanomolar; the y-axis indicates total cellular fluorescence divided by the number of nuclei per frame. AspA modified LNA are plotted as the left curve in blue, while the unmodified LNA is plotted as the right curve in red. The leftward shift of AspA modified LNA relative to unmodified indicates that AspA targeting enhances cellular uptake and retention under these conditions. FIG. 1B) Representative images from this experiment; AspA modified LNA was dosed at 200 nM, while unmodified LNA was dosed at 170 nM; blue represents nuclei staining by DAPI while green represents internalized LNA.

FIG. 2: Modification of MALAT1 targeting LNA with AspA enhances bioactivity as measured by qPCR.

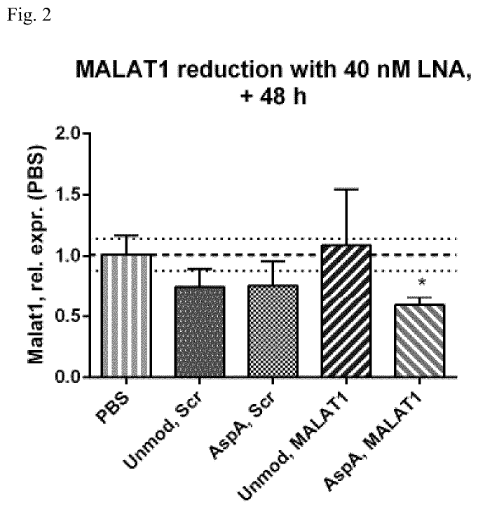

### DEFINITIONS

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one aspect, for the VL, the subgroup is subgroup kappa I as in Kabat et al., supra. In one aspect, for the VH, the subgroup is subgroup III as in Kabat et al., supra. [[Adapt as needed to refer to the actual subgroups of the VH/VLs of the invention]]

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-3), and three in the VL (CDR-L1, CDR-L2, CDR-L3). Exemplary CDRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system. [[Confirm with antibody engineering that claimed antibodies have CDRs defined by (a), (b) or (c), with Kabat (b) being the preferred definition. If the CDRs do not conform to the standard definitions, revise the definition of CDRs to include the claimed CDR residues.]]

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 BO.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, conjugates of the invention are used to delay development of a disease or to slow the progression of a disease.

Conjugation of the small molecule ligand to the cargo molecule may be performed using a variety of chemical linkers. For example, if the cargo molecule is a polypeptide, in particular an antibody, the small molecule ligand and polypeptide, in particular the antibody, may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52: 127-131 (1992); U.S. Pat. No. 5,208,020) may be used.

Covalent conjugation can either be direct or via a linker. In certain embodiments, direct conjugation is by formation of a covalent bond between a reactive group on one of the two portions of the small molecule ligand and a corresponding group or acceptor on the cargo molecule. In certain embodiments, direct conjugation is by modification (i.e., genetic modification) of one of the two molecules to be conjugated to include a reactive group (as non-limiting examples, a sulfhydryl group or a carboxyl group) that forms a covalent attachment to the other molecule to be conjugated under appropriate conditions. Methods for covalent conjugation of nucleic acids to proteins are also known in the art (i.e., photocrosslinking, see, e.g., Zatsepin et al. Russ. Chem. Rev. 74: 77-95 (2005)) Conjugation may also be performed using a variety of linkers. For example, a monovalent binding entity and a effector entity may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the effector entity upon delivery to the brain. For example, an acid-labile linker, peptidase-sensitive linker, photolabile linker, dimethyl linker or disulfidecontaining linker (Chari et al, Cancer Res. 52: 127-131 (1992); U.S. Pat. No. 5,208,020) may be used.

Pharmaceutical Compositions

In a further aspect, provided are pharmaceutical compositions comprising any of the conjugates provided herein, e.g., for use in any of the below therapeutic methods. In one aspect, a pharmaceutical composition comprises any of the conjugates provided herein and a pharmaceutically acceptable carrier. In another aspect, a pharmaceutical composition comprises any of the conjugates provided herein and at least one additional therapeutic agent, e.g., as described below.

Pharmaceutical compositions of a conjugate as described herein are prepared by mixing such conjugate having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or nonionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the conjugate, which matrices are in the form of shaped articles, e.g., films, or microcapsules.

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

The term “Antisense oligonucleotide” as used herein is defined as oligonucleotides capable of modulating expression of a target gene by hybridizing to a target nucleic acid, in particular to a contiguous sequence (a sub-sequence) on a target nucleic acid. The antisense oligonucleotides are not essentially double stranded and are therefore not siRNAs. Preferably, the antisense oligonucleotides of the present invention are single stranded.

An LNA antisense oligonucleotide is an antisense oligonucleotide which comprises at least one LNA nucleoside. In some embodiments the LNA antisense oligonucleotide is a LNA gapmer oligonucleotide.

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence and may optionally comprise further nucleotide(s), for example a nucleotide linker region which may be used to attach a functional group to the contiguous nucleotide sequence. The nucleotide linker region may or may not be complementary to the target nucleic acid.

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In some embodiments the modified nucleoside comprises a modified sugar moiety. The term modified nucleoside may also be used herein interchangeably with the term “nucleoside analogue” or modified “units” or modified “monomers”.

The term “modified intemucleoside linkage” is defined as generally understood by the skilled person as linkages other than phosphodiester (PO) linkages, that covalently couples two nucleosides together. Nucleotides with modified internucleoside linkage are also termed “modified nucleotides”. In some embodiments, the modified intemucleoside linkage increases the nuclease resistance of the oligonucleotide compared to a phosphodiester linkage. For naturally occurring oligonucleotides, the intemucleoside linkage includes phosphate groups creating a phosphodiester bond between adjacent nucleosides. Modified intemucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides.

In an embodiment, the oligonucleotide comprises one or more internucleoside linkages modified from the natural phosphodiester to a linkage that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant intemucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant intemucleoside linkages.

Modified intemucleoside linkages may be selected from the group comprising phosphorothioate, diphosphorothioate and boranophosphate. In some embodiments, the modified intemucleoside linkages are compatible with the RNaseH recruitment of the oligonucleotide of the invention, for example phosphorothioate, diphosphorothioate or boranophosphate.

In some embodiments the internucleoside linkage comprises sulphur (S), such as a phosphorothioate internucleoside linkage.

A phosphorothioate intemucleoside linkage is particularly useful due to nuclease resistance, beneficial pharmakokinetics and ease of manufacture. In some embodiments at least 50% of the intemucleoside linkages in the oligonucleotide, or contiguous nucleotide sequence thereof, are phosphorothioate, such as at least 60%, such as at least 70%, such as at least 80 or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

In some embodiments, the oligonucleotide comprises one or more neutral intemucleoside linkage, particularly a internucleoside linkage selected from phosphotriester, methylphosphonate, MMI, amide-3, formacetal or thioformacetal.

Further intemucleoside linkages are disclosed in WO2009/124238 (incorporated herein by reference). In an embodiment the internucleoside linkage is selected from linkers disclosed in WO2007/031091 (incorporated herein by reference). Particularly, the intemucleoside linkage may be selected from —O—P(O)₂—O—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(RH)—O—, O—PO(OCH₃)—O—, —O—PO(NRH)—O—, —O—PO(OCH₂CH₂S—R)—O—, —O—PO(BH₃)—O—, —O—PO(NHRH)—O—, —O—P(O)₂—NRH—, —NRH—P(O)₂—O—, —NRH—CO—O—, —NRH—CO—NRH—, and/or the internucleoside linker may be selected form the group consisting of: —O—CO—O—, —O—CO—NRH—, —NRH—CO—CH₂—, —OCH₂—CO—NRH—, —O—CH₂—CH₂—NRH—, —CO—NRH—CH₂—, —CH₂—NRHCO—, —O—CH₂—CH₂—S—, —SCH₂—CH₂—O—, —S—CH₂—CH₂—S—, —CH₂—SO₂—CH₂—, —CH₂—CO—NRH—, —O—CH₂—CH₂—NRH—CO—, —CH₂—NCH₃—O—CH₂—, where RH is selected from hydrogen and C₁₋₄ alkyl.

Nuclease resistant linkages, such as phosphothioate linkages, are particularly useful in oligonucleotide regions capable of recruiting nuclease when forming a duplex with the target nucleic acid, such as region G for gapmers, or the non-modified nucleoside region of headmers and tailmers. Phosphorothioate linkages may, however, also be useful in non-nuclease recruiting regions and/or affinity enhancing regions such as regions F and F′ for gapmers, or the modified nucleoside region of headmers and tailmers.

Each of the design regions may however comprise internucleoside linkages other than phosphorothioate, such as phosphodiester linkages, in particularly in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide—see WO2008/113832, incorporated herein by reference.

In an embodiment all the internucleoside linkages in the oligonucleotide are phosphorothioate and/or boranophosphate linkages. In some embodiments, all the internucleoside linkages in the oligonucleotide are phosphorothioate linkages.

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al. (2012) Accounts of Chemical Research vol 45 page 2055 and Bergstrom (2009) Current Protocols in Nucleic Acid Chemistry Suppl. 37 1.4.1).

The term “% complementary” as used herein, refers to the number of nucleotides in percent of a contiguous nucleotide sequence in a nucleic acid molecule (e.g. oligonucleotide) which, at a given position, are complementary to (i.e. form Watson Crick base pairs with) a contiguous nucleotide sequence, at a given position of a separate nucleic acid molecule (e.g. the target nucleic acid). The percentage is calculated by counting the number of aligned bases that form pairs between the two sequences, dividing by the total number of nucleotides in the oligonucleotide and multiplying by 100. In such a comparison a nucleobase/nucleotide which does not align (form a base pair) is termed a mismatch.

The term “fully complementary”, refers to 100% complementarity.

The term “Identity” as used herein, refers to the number of nucleotides in percent of a contiguous nucleotide sequence in a nucleic acid molecule (e.g. oligonucleotide) which, at a given position, are identical to (i.e. in their ability to form Watson Crick base pairs with the complementary nucleoside) a contiguous nucleotide sequence, at a given position of a separate nucleic acid molecule (e.g. the target nucleic acid). The percentage is calculated by counting the number of aligned bases that are identical between the two sequences, including gaps, dividing by the total number of nucleotides in the oligonucleotide and multiplying by 100. Percent Identity=(Matches×100)/Length of aligned region (with gaps).

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid. At physiological conditions Tm is not strictly proportional to the affinity (Mergny and Lacroix, 2003, Oligonucleotides 13:515-537). The standard state Gibbs free energy ΔG° is a more accurate representation of binding affinity and is related to the dissociation constant (Kd) of the reaction by ΔG°=-RTln(Kd), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

The term “target sequence” as used herein refers to a sequence of nucleotides present in the target nucleic acid which comprises the nucleobase sequence which is complementary to the oligonucleotide of the invention. In some embodiments, the target sequence consists of a region on the target nucleic acid which is complementary to the contiguous nucleotide sequence of the oligonucleotide of the invention. In some embodiments the target sequence is longer than the complementary sequence of a single oligonucleotide, and may, for example represent a preferred region of the target nucleic acid which may be targeted by several oligonucleotides of the invention.

The target sequence may be a sub-sequence of the target nucleic acid.

The oligonucleotide comprises a contiguous nucleotide sequence which is complementary to or hybridizes to the target nucleic acid, such as a sub-sequence of the target nucleic acid, such as a target sequence described herein.

The oligonucleotide comprises a contiguous nucleotide sequence of at least 8 nucleotides which is complementary to or hybridizes to a target sequence present in the target nucleic acid molecule. The contiguous nucleotide sequence (and therefore the target sequence) comprises of at least 8 contiguous nucleotides, such as 9, 10, 11, 12, 13, 14, 15, 16, 17, 18, 19, 20, 21, 22, 23, 24, 25, 26, 27, 28, 29 or 30 contiguous nucleotides, such as from 12-25, such as from 14-18 contiguous nucleotides.

The term a “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

The term “modulation of expression” as used herein is to be understood as an overall term for an oligonucleotide's ability to alter the amount of target gene expression when compared to the amount of target gene expression before administration of the oligonucleotide. Alternatively, modulation of expression may be determined by reference to a control experiment. It is generally understood that the control is an individual or target cell treated with a saline composition or an individual or target cell treated with a non-targeting oligonucleotide (mock). It may however also be an individual treated with the standard of care.

One type of modulation is an oligonucleotide's ability to inhibit, down-regulate, reduce, suppress, remove, stop, block, prevent, lessen, lower, avoid or terminate expression of NF-κB2 e.g. by degradation of mRNA or blockage of transcription.

A high affinity modified nucleoside is a modified nucleotide which, when incorporated into the oligonucleotide enhances the affinity of the oligonucleotide for its complementary target, for example as measured by the melting temperature (Tm). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

LNA nucleosides are modified nucleosides which comprise a linker group (referred to as a biradicle or a bridge) between C2′ and C4′ of the ribose sugar ring of a nucleotide. These nucleosides are also termed bridged nucleic acid or bicyclic nucleic acid (BNA) in the literature. Exemplary LNA nucleosides are disclosed in WO99/014226, WO00/66604, WO98/039352, WO2004/046160, WO00/047599, WO10036698, WO07090071, WO2010/036698 and WO11156202

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference).

The term gapmer as used herein refers to an antisense oligonucleotide which comprises a region of RNase H recruiting oligonucleotides (gap) which is flanked 5′ and 3′ by regions which comprise one or more affinity enhancing modified nucleosides (flanks or wings). Various gapmer designs are described herein. Headmers and tailmers are oligonucleotides capable of recruiting RNase H where one of the flanks is missing, i.e. only one of the ends of the oligonucleotide comprises affinity enhancing modified nucleosides. For headmers the 3′ flank is missing (i.e. the 5′ flank comprises affinity enhancing modified nucleosides) and for tailmers the 5′ flank is missing (i.e. the 3′ flank comprises affinity enhancing modified nucleosides).

The term LNA gapmer is a gapmer oligonucleotide wherein at least one of the affinity enhancing modified nucleosides is an LNA nucleoside.

The term mixed wing gapmer or mixed flank gapmer refers to a LNA gapmer wherein at least one of the flank regions comprise at least one LNA nucleoside and at least one nonLNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises only LNA nucleosides (e.g. 5′ or 3′) and the other flank (3′ or 5′ respectfully) comprises 2′ substituted modified nucleoside(s) and optionally LNA nucleosides.

### EXAMPLES

Biomacromolecules have diverse biochemical activities and unrivaled specificity and thus represent attractive drugs. While extracellular biomacromolecular therapies, especially recombinant proteins and therapeutic antibodies, have revolutionized multiple fields of medicine, the use of these modalities as marketed products with intracellular mechanisms of action remains limited to six oligonucleotide or oligonucleotide analogs (fomiversen, mipomersen, inotersen, patisiran, nusinersen, and eteplirsen) as well as the in situ administered viral modalities alipogene tiparvovec and voretigene neparvovec (1,7,8).

The use of biomacromolecules with intracellular mechanisms of action is primarily limited by two factors—systemic pharmacokinetics and intracellular sequestration by the endo-lysosomal system. Following endocytosis, the nascent endosome traffics through a progressively acidifying milieu of proteases, nucleases, and reducing enzymes.

The present invention overcomes both of these barriers by binding free cell surface thiols to tether biomacromolecules to the cell surface, enhancing local pharmacokinetics, and exploiting the natural recycling of these transmembrane proteins.

LNA function to reduce the mRNA transcript levels within cells by binding to mRNA in a complementary sequence specific manner, inducing target degradation by functioning as an RNAse H substrate or reducing protein levels by blocking ribosomal translation of mRNA.

We chemically conjugated AspA to a 5′ aminohexyl pendant from a phosphorothioated gapmer LNA using EDC/NHS chemistry and purified these molecules by HPLC. For cellular uptake studies, the parent LNA contained a fluorescent fluorescein isothiocyanate moiety which allowed us to localize this molecule inside cells by fluorescent microscopy.

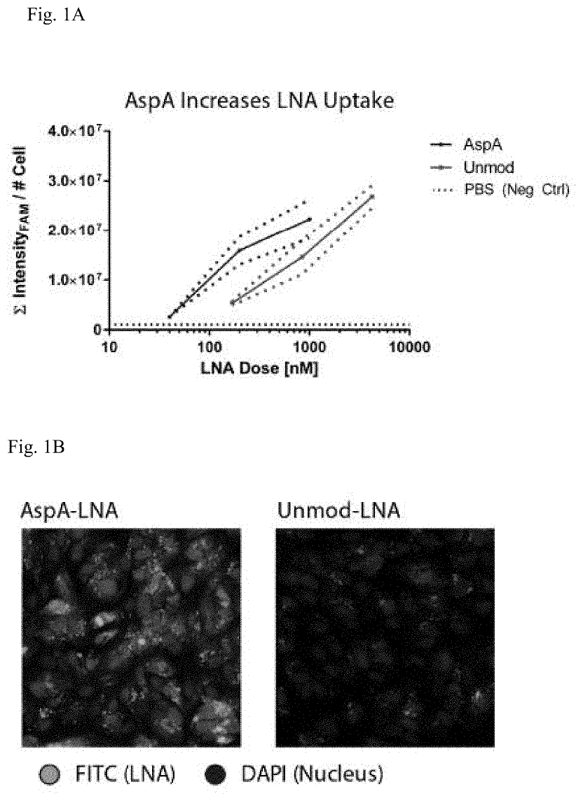

In cellular uptake studies, LNAs labeled with fluorescein isothiocyanate (FITC) were either used unmodified or conjugated to AspA. HCE-T cells were treated with a dilution series of stock LNAs at indicated doses and washed per “Cellular Uptake Methods”. When these images were quantified, it was found that LNA conjugation enhances cellular uptake, producing equivalent intracellular fluorescence as that of approximately 10-fold higher unmodified LNA dose, shown in FIG. 1A. AspA modified LNA are plotted as the left curve in blue, while the unmodified LNA is plotted as the right curve in red. The leftward shift of AspA modified LNA relative to unmodified indicates that AspA targeting enhances cellular uptake and retention under these conditions. Representative images from this experiment are shown as FIG. 1B, AspA modified LNA was dosed at 200 nM, while unmodified LNA was dosed at 170 nM; blue represents nuclei staining by DAPI while green represents internalized LNA. These results indicate AspA induces enhanced intracellular uptake, accumulation, and retention of LNA relative to unmodified LNA.

To test the activity of AspA LNA conjugates, HCE-T cells were treated with buffer or 40 nM LNAs, consisting of either scrambled, non-targeting sequence with or without AspA modification, or a potent LNA targeting the MALAT1 transcript with or without AspA targeting, FIG. 4. Both targeted and untargeted control sequence (“scr”) demonstrate modest offtarget effects of the MALAT1 transcript which were not statistically significant. The cells treated with unmodified LNA targeting MALAT1 mRNA showed a small increase to MALAT1 transcript levels, while the AspA LNA targeting MALAT1 induced statistically significant MALAT1 reduction. These results indicate AspA modification of LNA induces enhanced bioactivity for LNA molecules targeting the MALAT1 transcript. Because qPCR specifically measures the relative presence of mRNA, these results indicate that AspA modified LNAs retain strong RNAse H activity, and additionally indicate that AspA allows LNA to escape from endo-lysosomal entrapment and traffic to the nucleus, where RNAse H localizes.

Taken together, these data show that AspA conjugation to LNA enhances the bioactivity of LNAs by enhancing intracellular accumulation of LNAs and enables delivery of LNAs to the nucleus, where RNAse H activity degrades the target sequence (in this case, MALAT1) in a specific and potent manner.

Methods:
1. Structure of the targeting ligand 1,2-dithiolane-4-carboxylic acid, AspA

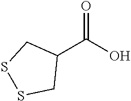

2. Reaction scheme for chemical conjugation of 1,2-dithiolane-4-carboxylic acid to hexylamino modified LNA

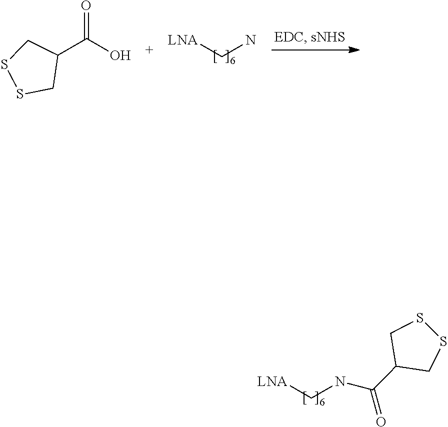

Phosphorothioated LNA were synthesized by standard phosphoroamidite chemistry and terminated with a 5′ hexylamino linker. The SML 1,2-dithiolane-4-carboxylic acid (asparagusic acid, AspA) was conjugated to the LNA amine via EDC/NHS coupling and purified by 2-propanol precipitation and HPLC. The LNA-AspA conjugate was applied to ARPE-19 and HCE-T cells for 30 minutes in PBS then washed with full serum media, simulating instillation via eye drops and challenge by tear proteins.

HCE-T Cell Culture

HCE-T cells were obtained from the Roche Non-Clinical Biorepository under a materials transfer agreement with Vanderbilt University. Cells were expanded in “expansion media:” Gibco DMEM/F12 with HEPES (cat. 31330) supplemented with 10% v/v FBS and no antibiotics. All cell manipulations were performed using collagen coated cultureware. PureCol Collagen I Solution (Advanced BioMatrix, cat. 5005) was diluted to 100 μg/mL in PBS and incubated for 1-2 h at room temperature and rinsed once with phosphate buffered saline. Plates were then used immediately or air dried for later use under sterile conditions.

Locked Nucleic Acids

Fully phosphorothioated LNA gapmers were purchased from Qiagen Sciences (Maryland, USA) with hexyl amino linkers. Some LNAs additionally had fluorescein modifications for cellular uptake studies. The small molecule targeting ligand 1,2-dithiolane-4-carboxylic acid (MedChemExpress, Cat. No. Cat. No.: HY-50730) was attached via a stable amide bond using 1-ethyl-3-(3-dimethylaminopropyl) carbodiimide hydrochloride (EDC, Sigma item 03449-1G)/N-hydroxysulfosuccinimide (s-NHS, Sigma item no. 56485-1G) chemistry9. Reaction mixtures were diluted with ultrapure water and the buffer was exchanged into ultrapure water using Amicon Ultra-0.5 mL centrifugal filters with a nominal molecular weight cutoff of 10 000 g/mol per manufacturer instructions, with two rounds of centrifugal concentration. The concentrated, buffer exchanged reaction mixture was diluted into 0.1 molar triethylammonium acetate buffer prepared from 1.0 M stock (Sigma Aldrich 90358-100ML). Purification was performed Oligos were analyzed using a Waters HPLC system equipped with a tunable photodiode array and single quadrupole mass detector. The stationary phase was a Waters XBridge Oligonucleotide BEH C18 Column, and the mobile phases were: A, 400 mM 1,1,1,3,3,3-Hexafluoro-2-propanol (HFIP) and 15 mM triethylamine (TEA) in water; B: 200 mM HFIP, 7.5 mM TEA, and 50% v/v methanol (MeOH) in water. The gradient was from 62% to 45% A, balance B in 17 minutes, followed by 13 minutes of equilibration at initial conditions. The purified fractions were lyophilized then reconstituted in sterile RNAse free water and stored at −20 C until final use.

Gene Downregulation Studies

HCE-T cells were plated in 96 well plates at 10,000 cells per well and grown to confluence in expansion media. At confluence, media was aspirated and cells were washed once with PBS, then LNAs were applied at indicated concentrations for 2 h then aspirated. Cells were washed once with expansion media before being incubated an additional 48 h in expansion media. After 48 h, mRNA was purified from cellular lysate using the Roche MagNA Pure 96 instrument according to manufacturer instructions. Purified RNA was quantified by UV absorbance and concentration normalized for cDNA synthesis using the iScript cDNA Synthesis Kit (Bio-Rad, Hercules, Calif., USA). Quantitative PCR was performed using TaqMan Fast Advanced Master Mix (ThermoFisher Scientific) and TaqMan primers targeting the model long noncoding RNA MALAT1 using ACTB as a reference gene according to manufacturer instructions.

Cellular Uptake Studies

HCE-T cells were plated in Corning 96-Well Half Area High Content Imaging Glass Bottom Microplates (Coming Cat. No 4580) at 1000 cells per well and grown to confluence in expansion media. At confluence, media was aspirated and cells were washed once with PBS, then fluorescent LNAs were applied at indicated concentrations for 2 h then aspirated. Cells were washed once with expansion media. Media was replaced with FluoroBrite DMEM (ThermoFisher Scientific) supplemented with 10% FBS, 15 mM HEPES buffer and Hoechst 33342. Cells were imaged on a Nikon A1 confocal microscope with appropriate excitation and emission filters. Total cellular fluorescence was measured using a MATLAB algorithm and normalized to number of nuclei.

### REFERENCES

1. Russell, S. et al. Efficacy and safety of voretigene neparvovec (AAV2-hRPE65v2) in patients with RPE65-mediated inherited retinal dystrophy: a randomised, controlled, open-label, phase 3 trial. Lancet 390, 849-860 (2017).
2. Adams, D. et al. Patisiran, an RNAi Therapeutic, for Hereditary Transthyretin Amyloidosis. N. Engl. J. Med. 379, 11-21 (2018).
3. Jansen, E. F. The isolation and identification of 2,2′-dithiolisobutyric acid from asparagus. J. Biol. Chem. 176, 657-64 (1948).
4. Abegg, D. et al. Strained Cyclic Disulfides Enable Cellular Uptake by Reacting with the Transferrin Receptor. J. Am. Chem. Soc. 139, 231-238 (2017).
5. Koshkin, A. A. et al. LNA (Locked Nucleic Acids): Synthesis of the adenine, cytosine, guanine, 5-methylcytosine, thymine and uracil bicyclonucleoside monomers, oligomerisation, and unprecedented nucleic acid recognition. Tetrahedron 54, 3607-3630 (1998).
6. Kumar, R. et al. The first analogues of LNA (locked nucleic acids): phosphorothioate-LNA and 2′-thio-LNA. Bioorg. Med. Chem. Lett. 8, 2219-22 (1998).
7. Stein, C. A. & Castanotto, D. FDA-Approved Oligonucleotide Therapies in 2017. Mol. Ther. 25, 1069-1075 (2017).
8. Akcea Therapeutics Press Release. Akcea and Ionis Receive FDA Approval of TEGSEDITM (inotersen) for the Treatment of the Polyneuropathy of Hereditary Transthyretin-Mediated Amyloidosis in Adults. (2018). Available at: https://ir.akceatx.com/news-releases/news-release-details/akcea-and-ionis-receive-fda-approval-tegseditm-inotersen. (Accessed: 25th Oct. 2018)
9. Hermanson, G. T. Zero-Length Crosslinkers. in Bioconjugate Techniques 259-273 (2013). doi:10.1016/B978—O—12-382239-0.00004-2

1. A conjugate comprising a small molecule targeting ligand and a cargo molecule having formula I: 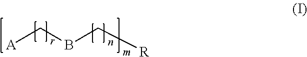
wherein A is selected from the group consisting of: 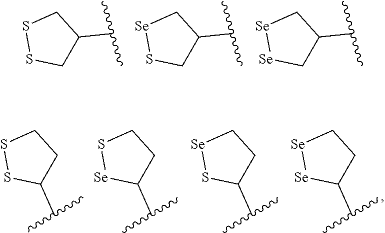
B is —C(O)—O— or —C(O)—N—,
n is selected from 0 or 1-6,
r is selected from 0 or 1-6
m is selected from 1-6, and
R is a cargo molecule.

2. The conjugate of claim 1, wherein the cargo molecule is selected from the group of peptides, polypeptides, oligonucleotides.

3. The conjugate of claim 1 or 2, wherein the cargo molecule is an antibody or an oligonucleotide.

4. The conjugate of claims 1-3, wherein the cargo molecule is a LNA oligonucleotide.

5. The conjugate of claim 3 or 4, wherein the small molecule targeting ligand is linked to the oligonucleotide at its 3′ end or 5′ end, preferably at its 5′ end.

6. The conjugate of claims 1-5, wherein B is —C(O)—N—.

7. The conjugate of claims 1-6, wherein r=0 and n=6.

8. The conjugate of claims 1-7, wherein m is selected from 1-4.

9. The conjugate of claims 1-8, wherein A is

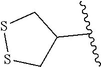 [SMILES: *C1CSSC1]

10. The conjugate of claims 1-9 having formula II:

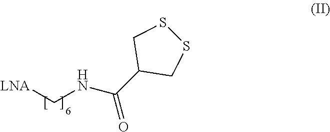

11. A pharmaceutical composition comprising a conjugate of claims 1-10 and pharmaceutically acceptable carrier.

12. The pharmaceutical composition of claim 11 being a topical composition for eye conditions.

13. A method of treating an individual having an eye condition comprising administering to the eye of the individual an effective amount of the conjugate of claims 1-10 or the pharmaceutical composition of claims 11 to 12.
